(19) United States
(12) Patent Application Publication
(10) Pub. No.: US 2020/0375348 A1
(43) Pub. Date: Dec. 3, 2020

(54) Toothbrush Sanitizer Holder/Case/Container

(71) Applicant: Jesus R. Amian, Waianae, HI (US)

(72) Inventor: Jesus R. Amian, Waianae, HI (US)

(21) Appl. No.: 16/423,222

(22) Filed: May 28, 2019

(51) Int. Cl. A46B 17/06; A61L 2/26; A61L 2/18

(52) U.S. Cl. CPC A46B 17/065; A46B 2200/1066; A61L 2/18; A61L 2/26

(57) ABSTRACT

In summary, my invention is a toothbrush holder that will sterilize and eliminate germs and bacteria on toothbrushes while being stored and not in use. The invention will have a base and a cover that is made of glass or plastic or ceramic. The base will be filled with the same material as the holder making the base heavy (center of gravity). The base cavity/pocket will be filled with mouthwash liquid, soaking the bristles of the toothbrush making it germ and bacteria free. The cover will eliminate air born germs and bacteria on the handle and rest of the toothbrush.

### BACKGROUND OF INVENTION

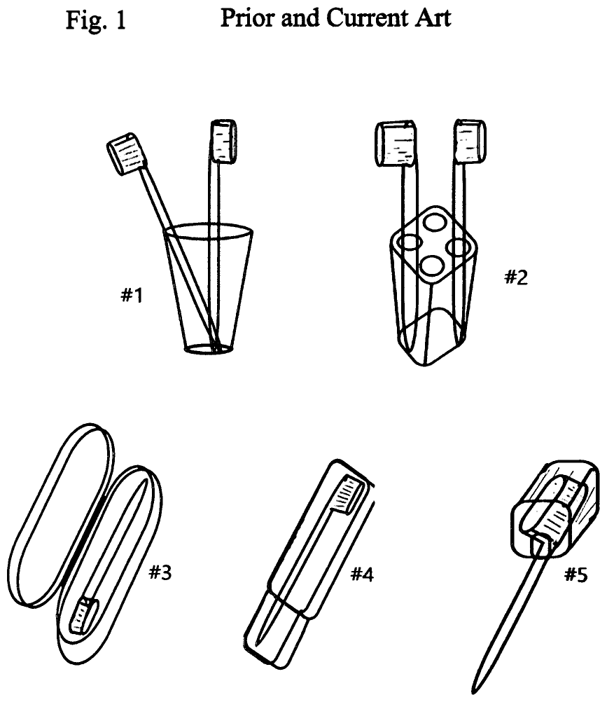

In prior and current art, a toothbrush holder is used to store toothbrushes while not in use. Toothbrush holders are mainly made of hard plastics, ceramic and glass. They come in many shapes and sizes. In FIG. 1, is a drawing of prior and current art. The problem with these holders is that bacteria and germs in the air settle onto a toothbrush and holder while the toothbrush is stored and not in use in the bathroom. There are current toothbrush holders that claim to sanitize toothbrush while not in use, by using UV light, sanitizer cases and storage containers to store their toothbrush.

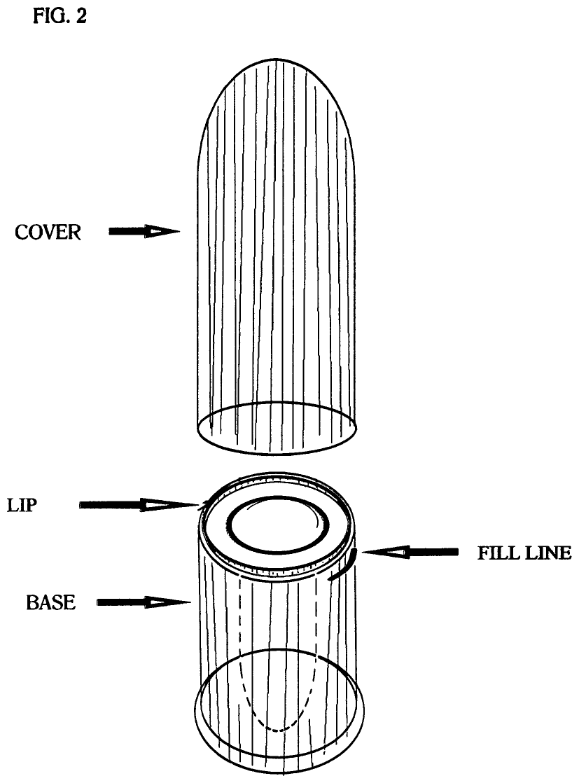

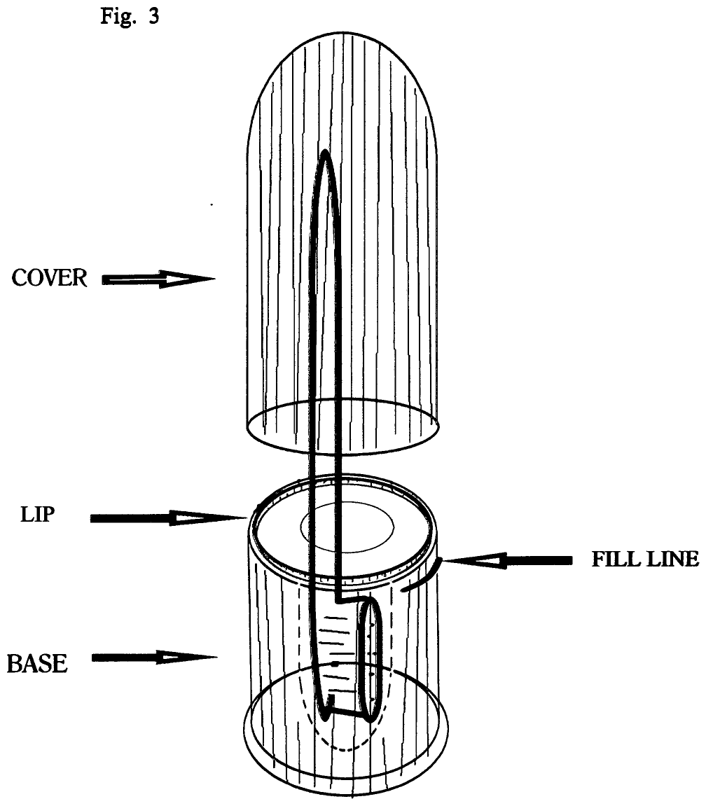

My invention is also a toothbrush holder that sanitizes/sterilizes toothbrushes. The difference with my invention versus other inventions is that my invention will eliminate bacteria and germs while a toothbrush is being stored and not in use. In FIGS. 2 and 3, shows a base and a cover. The base has a cavity/pocket that will be filled with sanitizer/sterilizer solution (mouthwash liquid) up to the fill line soaking the bristles of the toothbrush. This will sanitize bristle making toothbrush germ and bacteria free. And the cover will eliminate air born germs and bacteria from the handle and the rest of toothbrush.

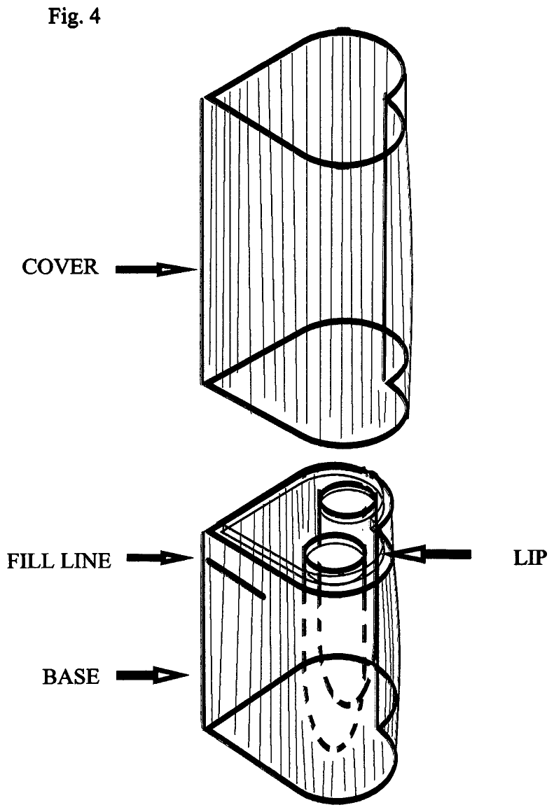

My toothbrush holder will be made with these materials: glass or plastic or ceramic. It will come in many different shape, size and color. For example, FIG. 2, cylinder design and FIG. 4, heart shape design are just 2 examples of many different shape and design styles for my invention. The base will be solid filled with the same material as the entire holder whether it's glass, plastic or ceramic. By filling the base, this will give the base weight and center of gravity making the toothbrush holder less likely to tip over and fall.

### DETAILED DESCRIPTION OF SEVERAL VIEWS OF THE DRAWINGS

FIG. 1 are frontal views of prior and current art.
1—Glass cup
2—Ceramic toothbrush holder
3—Plastic case
4—Plastic case
5—Plastic case only for toothbrush bristles

FIG. 2 is a frontal view of invention
1—Cylinder shape
   a. Base—cavity/pocket
   b. Cover
   c. Fill line
   d. Lip

FIG. 3 is a frontal view of invention: purpose and function
1—Cylinder shape
   a. Base—cavity/pocket
   b. Cover
   c. Fill line
   d. Lip
   e. Toothbrush

FIG. 4 is a frontal view of invention: example of different shape and style
1—Heart shape
   a. Base—cavity/pocket
   b. Cover
   c. Fill line
   d. Lip

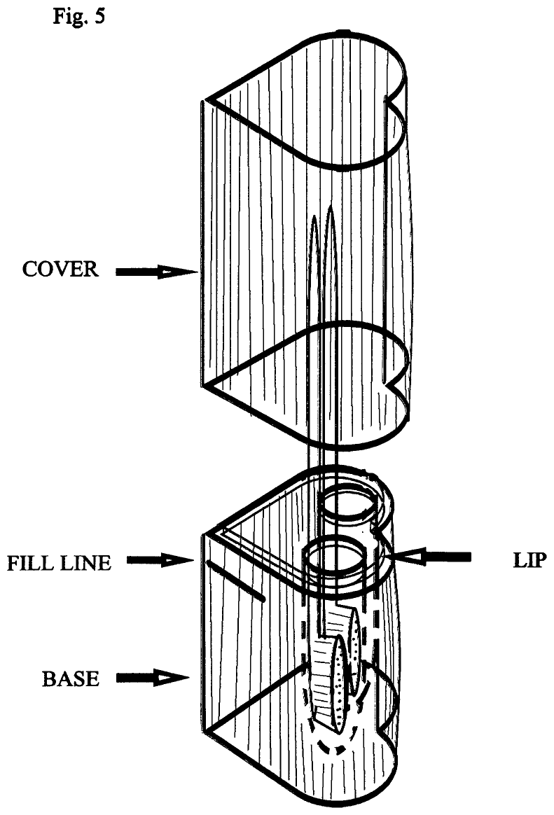

FIG. 5 is a frontal view of invention: purpose and function
1—Heart shape
   a. Base—cavity/pocket
   b. Cover
   c. Fill line
   d. Lip
   e. Toothbrush

### DETAILED DESCRIPTION OF THE INVENTION

My invention is a toothbrush holder that will reduce and eliminate germs and bacteria on toothbrushes while being stored and not in use. The invention is made of a base and a cover.

The base will hold mouthwash liquid (sanitizer) and the cover will cover the rest of the toothbrush eliminating germs and bacteria from the rest of the toothbrush while being stored.

The base and cover will be made with materials such as glass, plastic or ceramic. The base will be filled with the same material as the rest of the holder making the base heavy (center of gravity).

Bathroom sinks, toilets and floors are all areas where bacteria and germs are present. Because toothbrushes are stored near toilets and sinks it's also subject to these germs and bacteria.

Inventions, such as, toothbrush holders are used to store toothbrushes while not in use. The problem with these toothbrush holders is that the toothbrush and holder are loaded with germs and bacteria, just minutes after washing and rinsing toothbrush. Germs and bacteria in the air settles on toothbrush and multiply while toothbrush is being stored in toothbrush holders while sitting in the bathroom.

According to reference articles and images; Bing.com/images title: Germs and Bacteria in the bathroom. 1. Fact, “flushing a toilet send bacteria two meters around bathroom.” 2. Fact, “average bacteria on a toilet seat: 50 bacteria per sq. in.” 3. Tip, “make sure your toothbrush is 6 ft. away from the toilet and close the lid before flushing.”

My invention is also a toothbrush holder. The difference with my invention versus prior and current art is that my invention will sanitize/sterilize toothbrush while being stored and not in use.

FIG. 2 is a frontal view of the invention
1—Cover
   a. Is to cover entire toothbrush while being stored
   b. Reduce and eliminate germs and bacteria on entire toothbrush
   c. Materials are glass, plastic or ceramic
2—Base
   a. Will be filled in with the same material used for entire invention making the base solid and heavy giving it a heavy center of gravity
   b. Will have a cavity/pocket for toothbrush and sanitizer liquid (mouthwash)
   c. Materials are glass, plastic or ceramic for base
3—Lip
   a. Is to hold cover in place (stationary)
4—Fill Line
   a. In the base there is a cavity/pocket that will be filled with sanitizer liquid (mouthwash)

### REFERENCE ARTICLES

1—Bing.com/images
   Title: Images of bathroom facts—Germs and Bacteria in bathroom
   a. Flushing a toilet send bacteria up to two meters around bathroom
   b. Different types of germs found in bathroom
      1—Fecal Coliform Bacteria
      2—E. Coil
      3—Mold Spores
      4—Strep
   c. Where you'd find it
      1—Toothbrush around a toilet
      2—On a towel
      3—On a shower curtain
      4—In around bath or sink
      5—In damp places
      6—On makeup bristles
   d. What it does & what it can cause
      1—Can cause diarrhea
      2—Vomiting
      3—Stomach cramps
      4—Fever
      5—Can cause stiffness
      6—Throat irritation
      7—Coughing or sneezing
      8—Eye irritation
      9—Strep throat
      10—Scarlet fever
      11—Impetigo
   e. Toilet seat: Average bacteria on a toilet seat: 50 bacteria per sq. in.
   f. Hygiene fact
      1—Most germs can stay alive on your hands for 3 hours
   g. Where do you keep toothbrush?
      1—Make sure your toothbrush is 6 ft. away from the toilet and close the lid before flushing
   2—Pennmedical.org
      Title: Hidden Germs: 8 bath room hygiene mistakes your making
      Date: Oct. 23, 2017
   3—Webmd.com
      Title: Germs in the bathroom
      Author Denise Mann
   4—Reader's Digest.com
      Title: The germiest spots in your bathroom (Hint: Your toilet isn't #1)
      Author Marissa Laliberte
   5—Time.com
      Title: The Germiest Place in your bathroom isn't your toilet
      Author: Julissa Trevino
      Date: Apr. 25, 2019

1. A toothbrush holder having a base and a cover that sits on the base, said base having a fill line and configured to hold sanitizer liquid up to said fill line for immersing a head portion of a toothbrush therein while being stored and said cover covering the rest of the toothbrush for eliminating germs and bacteria from the rest of the toothbrush while being stored, wherein said base is made so as to have a greater weight of material than the cover in order to provide a lowered center of gravity so that the toothbrush holder is less likely to tip or fall.

2. The toothbrush holder of claim 1, wherein the said base and said cover are configured to hold up two toothbrushes.
